(19) United States
(12) Patent Application Publication
(10) Pub. No.: US 2015/0150767 A1
(43) Pub. Date: Jun. 4, 2015

(54) Compositions Containing Fatty Alcohols, Cationic Surfactants And N-Acyl-N-Methylglucamines

(71) Applicant: CLARIANT INTERNATIONAL LTD., Muttenz (CH)

(72) Inventors: Peter Klug, Grossostheim (DE); Gerd Dahms, Duisburg (DE); Carina Mildner, Frankfurt am Main (DE)

(73) Assignee: CLARIANT FINANCE (BVI) LIMITED, Tortola (VG)

(21) Appl. No.: 14/403,049

(22) PCT Filed: May 29, 2013
(86) PCT No.: PCT/EP2013/061106

§ 371 Date: Nov. 21, 2014

(30) Foreign Application Priority Data
May 30, 2012 (DE) 10 2012 010 699.8
Apr. 20, 2013 (DE) 10 2013 006 878.9

(51) Int. Cl. A61K 8/44; A61K 8/34; A61K 8/41; A61Q 5/06; A61Q 19/00; A61Q 5/12; A61Q 5/04; A61K 8/06; A61Q 5/00

(52) U.S. Cl. CPC A61K 8/44; A61K 8/062; A61K 8/342; A61K 8/416; A61Q 5/00; A61Q 19/00; A61Q 5/12; A61Q 5/04; A61Q 5/065; A61K 2800/43; A61K 2800/48

(57) ABSTRACT

A composition which contains a) one or more N-methyl-N-acylglucamines, at least 80 wt.-% of the N-methyl-N-acylglucamines having a saturated or unsaturated C₁₆-, C₁₇- and/or C₁₈-acyl group, b) one or more fatty alcohols, c) one or more cationic surfactants, d) optionally other additives, and e) water is suitable for producing cosmetic, dermatological and pharmaceutical emulsions, especially for use in hair care products.

The invention relates to compositions, preferably cosmetic, dermatological and pharmaceutical compositions, comprising fatty alcohols, cationic surfactants and particular N-acyl-N-methylglucamines, to the use thereof for skincare and haircare, and to the use of the N-acyl-N-methylglucamines for thickening and improving the care action of cationic emulsions.

Care products for skincare and haircare comprising N-acyl-N-methylglucamines, fatty alcohols and quaternary ammonium compounds having at least one long-chain alkyl or alkenyl group, for example behenyltrimethylammonium chloride, stearyltrimethylammonium chloride, lauryltrimethylammonium chloride, cetyltrimethylammonium chloride, distearyldimethylammonium chloride, are known.

Also known are cosmetic compositions comprising fatty acid N-alkylpolyhydroxyalkylamides and fatty alcohols.

WO 97/47284 proposes novel cosmetic formulations for use in the haircare and skincare sector, comprising (a) ester quats and (b1) sorbitan esters, (b2) polyol poly-12-hydroxystearates and/or (b3) glycerides and optionally (c1) alkyl and/or alkenyl oligoglycosides and/or (c2) fatty acid N-alkylpolyhydroxyalkylamides. The products feature improved softness of the hair and a particularly pleasant skin feel. There is specific disclosure of examples with fatty acid N-alkylpolyhydroxyalkylamides derived from coconut fatty acid.

WO 94/21226 discloses detergent mixtures comprising polyhydroxy fatty acid amides, monomeric cationic surfactants, fatty alcohols and optionally oil bodies, hair treatment products comprising these mixtures and the use of the mixtures for production of hair treatment products. There is specific disclosure of examples with C12/C14 fatty acid N-alkylpolyhydroxyalkylamides.

Fine, storage-stable emulsions comprising, inter alfa, fatty acid N-alkylpolyhydroxyalkylamides and fatty alcohols are known, for example, from WO 97/06870. Preference is given here to fatty acid N-alkylpolyhydroxyalkylamides based on lauric acid or C12/C14 coconut fatty acid.

WO 96/27366 discloses cosmetic and pharmaceutical compositions comprising, as 0/W emulsifiers, fatty acid N-alkylglucamides in combination with fatty alcohols as coemulsifiers. There is no pointer to a conditioning effect in haircare.

Even though good results have already been achieved with the known systems, there is a great deal of room for improvement, especially with regard to use in cosmetic compositions for haircare and skincare.

The object was thus to develop surfactant systems which have good formulability, have higher stabilities, exhibit improved conditioning effects, for example bring about better combability of wet and dry hair, reduce static discharge, improve the feel, and achieve shine and protection of hair color. More particularly, there is a need for active ingredients which, in spite of good biodegradability, have excellent conditioning properties and additionally good skin compatibility. The development of active ingredients should also include the possibility of providing the active ingredients in the form of compositions, in which case they may already have been formulated.

It has been found that this object is achieved by compositions comprising particular N-acyl-N-methylglucamines, fatty alcohols and cationic surfactants.

The invention therefore provides a composition comprising
a) one or more N-methyl-N-acylglucamines, where at least 50% by weight of the N-methyl-N-acylglucamines have a saturated or unsaturated C₁₆-, C₁₇- and/or C₁₈-acyl group,
b) one or more fatty alcohols,
c) one or more monomeric cationic surfactants,
d) optionally further additives and
e) water.

Inventive compositions have improved thermal stability, improved viscosity values and, in the case of haircare products, a good hair feel. The combination of surfactants a) to c) is especially suitable for thickening cationic emulsions.

The inventive compositions preferably comprise
(a) 0.1% to 5% by weight of component (a),
(b) 0.1% to 10% by weight of component (b),
(c) 0.1% to 5% by weight of component (c),
(d) 0% to 30% by weight of component (d) and
(e) 99.8% to 50% by weight of component (e),
   where the sum total of components (a), (b), (c), (d) and (e) is 100% by weight.

The N-methyl-N-acylglucamines used in accordance with the invention, in which glucamine is an N-1-deoxysorbityl group, contain at least 50% by weight of N-methyl-N-acylglucamines containing a saturated or unsaturated C₁₆-C₁₈-acyl group. Preferably, the proportion of N-methyl-N-acylglucamines containing a saturated or unsaturated C₁₆-C₁₈-acyl group is at least 80% by weight, more preferably at least 90% by weight. In addition, the N-methyl-N-acylglucamines used in accordance with the invention contain small proportions of N-methyl-N-acylglucamines derived from short-chain and/or long-chain fatty acids, especially those containing C₁-C₄-acyl, C₆-, C₈-, C₁₀-, C₁₂-, C₁₄- and/or C₂₀-acyl.

Especially preferred as component a) are saturated N-methyl-N-acylglucamines of the formula (I) where the R^{a}CO acyl radical is derived from palmitic acid, stearic acid, oleic acid or linoleic acid:

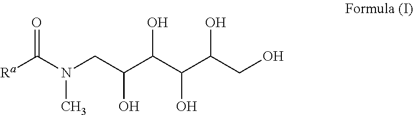

More preferably, the proportion of component a) in the inventively composition is 0.2% by weight to 3.0%, most preferably 0.5% by weight to 2%.

The N-methyl-N-acylglucamines may, as described in EP 0 550 637 A1, be prepared by reacting the corresponding fatty acid esters or fatty acid ester mixtures with N-methylglucamine in the presence of a solvent having hydroxyl groups or alkoxy groups. Suitable solvents are, for example, C₁-C₄ monoalcohols, ethylene glycol, 1,2-propylene glycol, glycerol and alkoxylated alcohols. Preference is given to 1,2-propylene glycol. N-Methylglucamine can, as likewise described in EP 0 550 637 A1, be obtained by reductive amination of glucose with methylamine.

Suitable fatty acid esters which are reacted with the N-methylglucamines to give N-methyl-N-acylglucamines are generally the methyl esters, which are obtained by transesterification from natural fats and oils, for example the triglycerides.

Suitable raw materials for the preparation of the fatty acid methyl esters are, for example, coconut oil or palm oil.

The invention also provides for the use of one or more of the N-methyl-N-acylglucamines used in accordance with the invention for thickening emulsions.

The inventive compositions contain one or more fatty alcohols b), i.e. unbranched or branched monoalcohols having an alkyl group having 8 to 22 carbon atoms, and preferably, based on the overall composition, from 0.1% to 10.0% by weight of one or more of these substances. Preference is therefore further given to inventive compositions comprising one or more unbranched or branched monoalcohols having an alkyl group having 8 to 22 carbon atoms, and preferably, based on the overall composition, from 0.1% to 10.0% by weight of one or more of these substances.

Useful monoalcohols of this kind preferably include lauryl alcohol, stearyl alcohol, cetyl alcohol, Guerbet alcohol, behenyl alcohol and mixtures thereof.

The invention also provides for the use of one or more N-methyl-N-acylglucamines used in accordance with the invention in combination with at least one fatty alcohol (b), where the ratio of components a:b is 1:9 to 5:5.

The inventive composition further comprises at least one monomeric cationic surfactant as component c).

Preferably, the proportion of component c) in the inventive composition is 0.1% by weight to 3.0%, more preferably 0.5% by weight to 2%.

Suitable cationic surfactants are, for example, substituted or unsubstituted, straight-chain or branched quaternary ammonium salts of the R¹N(CH₃)₃X, R¹R²N(CH₃)₂X, R¹R²R³N(CH₃)X or R¹R²R³R⁴NX type. The R¹, R², R³ and R⁴ radicals may preferably each independently be unsubstituted alkyl having a chain length between 8 and 24 carbon atoms, especially between 10 and 18 carbon atoms, hydroxyalkyl having 1 to 4 carbon atoms, phenyl, C₂- to C₁₈-alkenyl, C₇- to C₂₄-aralkyl, (C₂H₄O)ₓH where x is from 1 to 3, alkyl radicals containing one or more ester groups, or cyclic quaternary ammonium salts. X is a suitable anion. Preference is given to (C₈-C₂₂)-alkyltrimethylammonium chloride or bromide, more preferably cetyltrimethylammonium chloride or bromide, di-(C₈-C₂₂)-alkyldimethyl-ammonium chloride or bromide, (C₈-C₂₂)-alkyldimethylbenzylammonium chloride or bromide, (C₈-C₂₂)-alkyldimethylhydroxyethylammonium chloride, phosphate, sulfate, lactate, more preferably distearyldimethylammonium chloride, di-(C₈-C₂₂)-alkylamidopropyltrimethylammonium chloride and methosulfate.

Preferred cationic surfactants are compounds of the formula (II)

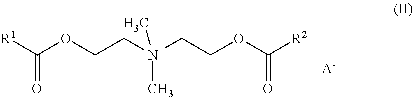

in which
R¹CO and R²CO are each independently linear or branched saturated acyl groups having 18 to 24 carbon atoms,
A⁻ is a counterion
and the total amount of C₁₈₋₂₃-alkyl-COO— groups, preferably the total amount of C₁₉₋₂₃-alkyl-COO— groups, based on all the R¹COO— and R²COO— groups, is 40.0% by weight or more, preferably 42.0% by weight or more.

The compounds of the formula (II) in which R¹CO and R²CO are each independently linear or branched saturated acyl groups having 18 to 24 carbon atoms can also be described in such a way that R¹ and R² in formula (II) are each independently linear or branched alkyl groups having 17 to 23 carbon atoms.

Preferably, the R¹ and R² radicals in the compounds of the formula (II) are each independently linear alkyl groups having 17 to 23 carbon atoms.

In a preferred embodiment of the invention, the total amount of C₁₉-alkyl-COO—, C₂₁-alkyl-COO— and C₂₃-alkyl-COO— groups, based on all the R¹COO— and R²COO— groups, is 40.0% by weight or more and preferably 42.0% by weight or more.

In a particularly preferred embodiment of the invention, the total amount of C₁₉-alkyl-COO— and C₂₁-alkyl-COO— groups, based on all the R¹COO— and R²COO— groups, is 40.0% by weight or more and preferably 42.0% by weight or more.

In an especially preferred embodiment of the invention, the total amount of C₂₁-alkyl-COO— groups, based on all the R¹COO— and R²COO— groups, is 40.0% by weight or more and preferably 42.0% by weight or more.

In an exceptionally preferred embodiment of the invention, it is a feature of the inventive compositions that several compounds of the formula (II) are present therein, and the amount of C₁₇-alkyl-COO— groups in the compounds of the formula (II), based on all the R¹COO— and R²COO— groups, is from 1.0% to 45.0% by weight, and the amount of C₁₉-alkyl-COO— groups in the compounds of the formula (II), based on all the R¹COO— and R²COO— groups, is from 1.0% to 15.0% by weight, and the amount of C₂₁-alkyl-COO— groups in the compounds of the formula (II), based on all the R¹COO— and R²COO— groups, is from 42.0% to 94.0% by weight.

In an extremely preferred embodiment of the invention, it is a feature of the inventive compositions that several compounds of the formula (II) are present therein, and the amount of C₁₇-alkyl-COO— groups in the compounds of the formula (II), based on all the R¹COO— and R²COO— groups, is from 41.0% to 45.0% by weight, and the amount of C₁₉-alkyl-COO— groups in the compounds of the formula (II), based on all the R¹COO— and R²COO— groups, is from 9.0% to 13.0% by weight, and the amount of C₂₁-alkyl-COO— groups in the compounds of the formula (II), based on all the R¹COO— and R²COO— groups, is from 42.0% to 46.0% by weight.

In a further extremely preferred embodiment of the invention, it is a feature of the inventive compositions that several compounds of the formula (II) are present therein, and the amount of C₁₇-alkyl-COO— groups in the compounds of the formula (II), based on all the R¹COO— and R²COO— groups, is from 1.0% to 6.0% by weight, and the amount of C₁₉-alkyl-COO— groups in the compounds of the formula (II), based on all the R¹COO— and R²COO— groups, is from 12.0% to 15.0% by weight, and the amount of C₂₁-alkyl-COO— groups in the compounds of the formula (II), based on all the R¹COO— and R²COO— groups, is from 79.0% to 84.0% by weight.

In a further extremely preferred embodiment of the invention, it is a feature of the inventive compositions that several compounds of the formula (II) are present therein, and the amount of C₁₇-alkyl-COO— groups in the compounds of the formula (II), based on all the R¹COO— and R²COO— groups, is from 1.0% to 6.0% by weight, and the amount of C₁₉-alkyl-COO— groups in the compounds of the formula (II), based on all the R¹COO— and R²COO— groups, is from 1.0% to 6.0% by weight, and the amount of C₂₁-alkyl-COO— groups in the compounds of the formula (II), based on all the R¹COO— and R²COO— groups, is from 90.0% to 94.0% by weight.

In a further preferred embodiment of the invention, the amount of C₂₃-alkyl-COO— groups in the compounds of the formula (II), based on all the R¹COO— and R²COO— groups, in the inventive compositions is up to 3.0% by weight, preferably from 0.5% to 2.5% by weight and more preferably from 1.0% to 2.0% by weight.

In a further extremely preferred embodiment of the invention, it is a feature of the inventive compositions that several compounds of the formula (II) are present therein, and the amount of C₁₇-alkyl-COO— groups in the compounds of the formula (II), based on all the R¹COO— and R²COO— groups, is from 1.0% to 5.0% by weight, and the amount of C₁₉-alkyl-COO— groups in the compounds of the formula (1), based on all the R¹COO— and R²COO— groups, is from 6.0% to 10.0% by weight, and the amount of C₂₁-alkyl-COO— groups in the compounds of the formula (II), based on all the R¹COO— and R²COO— groups, is from 86.0% to 90.0% by weight. Among these inventive compositions, preference is in turn given to those in which the amount of C₂₃-alkyl-COO— groups in the compounds of the formula (II), based on all the R¹COO— and R²COO— groups, is from 1.0% to 5.0% by weight.

In a further preferred embodiment of the invention, the inventive compositions comprise one or more compounds of the formula (II) in which R¹ and R² are each linear or branched, preferably linear, alkyl groups having 21 carbon atoms.

Likewise preferred as cationic surfactants are alkyltrimethylammonium chlorides or methosulfates of the formula (III)

CₙH₂ₙN⁺(CH₃)₃X⁻ (III)
where n is preferably 16, 18, 20 or 22 and X⁻ is a chloride ion or methosulfate ion.

Further preferred cationic surfactants are surfactants of the formula (IV)

[R¹CO—NH—CH₂—CH₂—CH₂—NR²R³R⁴]X⁻ (IV)

where R¹CO is a linear or branched, saturated or unsaturated acyl radical having 16 to 32 carbon atoms, preferably a saturated, unbranched acyl radical having 18-22 carbon atoms, R² and R³ are each methyl, R⁴ is H or methyl and X⁻ is an anion preferably selected from chloride, methosulfate, lactate, citrate, glutamate and glycolate.

The invention further provides for the use of components a) as thickeners for emulsions, preferably for thickening cationic emulsions.

In the case of this use, components a) and b) are preferably used in a weight ratio of 1:9 to 5:5.

As well as components a), b), c) and water e), the inventive compositions optionally comprise further additives (d).

To improve the consistency of the composition, it may be advantageous to add one or more short-chain monoalcohols thereto.

In a further preferred embodiment of the invention, the composition therefore comprises one or more monoalcohols having an alkyl group having 1 to 7 carbon atoms, and preferably, based on the overall composition, from 0% to 10.0% by weight of one or more of these substances.

Preference is given to using, as monoalcohols, ethanol, propanol, isopropanol, butanol, isobutanol and t-butanol, more preferably isopropanol.

In a further preferred embodiment of the invention, the composition comprises one or more polyols having 3 to 12 carbon atoms, and preferably, based on the overall composition, from 0% to 10.0% by weight of one or more of these substances.

Preferred polyhydric alcohols, i.e. polyols, are 1,2-propylene glycol, 1,3-propylene glycol, pentanediol, hexanediol, heptanediol, octanediol, nonanediol, decanediol, undecanediol, dodecanediol, diglycerol, triglycerol, dipropylene glycol, tripropylene glycol, sorbitol, xylitol, mannitol and/or mixtures thereof. Particularly preferred polyhydric alcohols, i.e. polyols, are 1,2-propylene glycol, 1,3-propylene glycol, pentane-1,5-diol, pentane-1,2-diol, hexane-1,6-diol, hexane-1,2-diol, heptane-1,7-diol, heptane-1,2-diol, octane-1,8-diol, octane-1,2-diol, nonane-1,9-diol, nonane-1,2-diol, decane-1,10-dial, decane-1,2-diol, undecane-1,11-diol, undecane-1,2-diol, dodecane-1,12-diol, dodecane-1,2-diol, diglycerol, triglycerol, dipropylene glycol, tripropylene glycol, sorbitol, xylitol, mannitol and/or mixtures thereof.

In a further preferred embodiment of the invention, the inventive compositions comprise one or more fatty acids of the formula RCOOH in which RCO are linear or branched acyl groups having 8 to 24 carbon atoms, and preferably, based on the overall composition, from 0.1% to 5.0% by weight of one or more of these substances.

The conditioning effect of the inventive compositions can be enhanced by addition of N-modified silicones.

In a further preferred embodiment of the invention, the inventive compositions comprise alkylmethicones, alkyldimethicones or one or more amodimethicones. Amodimethicones are siloxanes polymers grafted with amino-functional groups. Amodimethicones are, for example, under the brand name Dow Corning 2-8566 Amino Fluid (Dow Corning Corporation), Mirasil ADME (Rhodia), SilCare® Silicone SEA (Clariant) or Wacker-Belsil ADM 1100 (Wacker Chemie AG), have a molecular weight between 800 and 260 000 g/mol and correspond in general terms to the formula (V)

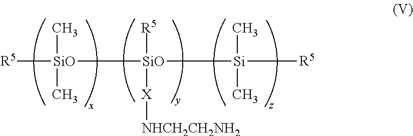

in which
R⁵ is —OH or —CH₃,
X is a linear or branched C₁-C₆-alkylene group and
x, y and z are each independently a number from 1 to 5500, preferably from 50 to 500.

In a particularly preferred embodiment of the invention, the inventive compositions comprise one or more of the abovementioned compounds of the formula (V) and preferably, based on the overall composition, 0.1% to 5.0% by weight of one or more compounds of the formula (V).

Compatibility with further ingredients, and also the skin feel and antistatic effect of the composition, can be improved through addition of one or more compounds of the formula (VI)

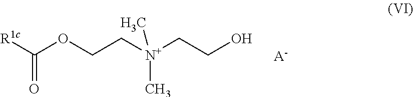

in which
R^{1c}CO is a linear or branched, preferably a linear, further preferably a saturated, acyl group having 18 to 24 carbon atoms, preferably having 18 to 22 carbon atoms, and
A⁻ is a counterion.

In a further preferred embodiment of the invention, the inventive compositions comprise one or more compounds of the formula (VI), preferably, based on the overall composition, in a proportion of 0.1% to 5.0% by weight.

The counterion A⁻ of the formula (VI) is preferably selected from chloride, bromide, methosulfate (MeSO₄⁻), tosylate, phosphate, sulfate, hydrogen-sulfate, lactate and citrate, and is more preferably selected from chloride and methosulfate.

In a further preferred embodiment of the invention, the inventive compositions are emulsions.

The cosmetic, dermatological and pharmaceutical compositions may comprise, as further auxiliaries and additives of component (d), all the customary surfactants, oil bodies, cationic polymers, film formers, thickeners and gelating agents, superfatting agents, antimicrobial and biogenic active ingredients, moisture donors, stabilizers, preservatives, pearlizing agents, dyes and fragrances.

Further surfactants used may be nonionic, amphoteric and/or zwitterionic surfactants.

Preferred nonionic surfactants are fatty alcohol ethoxylates (alkyl polyethylene glycols); alkylphenol polyethylene glycols; fatty amine ethoxylates (alkylamino polyethylene glycols); fatty acid ethoxylates (acyl polyethylene glycols); polypropylene glycol ethoxylates (Pluronics®); fatty acid alkanolamides (fatty acid amide polyethylene glycols); sucrose esters; sorbitol esters and sorbitan esters and the polyglycol ethers thereof, and also C₈-C₂₂-alkyl polyglucosides.

The amount of the nonionic surfactants in the inventive compositions (for example in the case of rinse-off products) is preferably in the range from 0.1% to 10.0% by weight, more preferably from 0.5% to 5.0% by weight and especially preferably from 1.0% to 3.0% by weight.

In addition, the inventive compositions may comprise amphoteric surfactants. These may be described as derivatives of long-chain secondary or tertiary amines which have an alkyl group having 8 to 18 carbon atoms and in which a further group is substituted by an anionic group which imparts water solubility, for example by a carboxyl, sulfate or sulfonate group. Preferred amphosurfactants are N—(C₁₂-C₁₈)alkyl β-aminopropionates and N—(C₁₂-C₁₈)alkyl β-iminodipropionates in the form of alkali metal and mono-, di- and trialkylammonium salts. Suitable further surfactants are also amine oxides. These are oxides of tertiary amines having a long-chain group of 8 to 18 carbon atoms and two usually short-chain alkyl groups having 1 to 4 carbon atoms. Preference is given here, for example, to the C₁₀- to C₁₈-alkyldimethylamine oxides and fatty acid amidoalkyl dimethylamine oxides.

A further preferred group of surfactants is that of betaine surfactants, also called zwitterionic surfactants. These contain, in the same molecule, a cationic group, especially an ammonium group, and an anionic group, which may be a carboxylate group, sulfate group or sulfonate group. Suitable betaines are preferably alkyl betaines such as cocobetaine or fatty acid alkylamidopropyl betaines, for example cocoacylamidopropyl dimethyl betaine, or C₁₂-C₁₈ dimethylaminohexanoates or C₁₀-C₁₈ acylamidopropane dimethyl betaines.

The amount of the amphoteric surfactants and/or betaine surfactants is preferably from 0.5% to 20.0% by weight and more preferably from 1.0% to 10.0% by weight.

Preferred surfactants are cocoamidopropyl betaine, alkyl betaines such as cocobetaine, sodium cocoylglutamate and lauroamphoacetate.

In a preferred embodiment, the inventive compositions additionally comprise, as foam enhancers, cosurfactants from the group of the alkyl betaines, alkylamido betaines, aminopropionates, aminoglycinates, imidazolinium betaines and sulfo betaines, amine oxides, fatty acid alkanolamides and polyhydroxy amides.

The oil bodies may advantageously be selected from the groups of the triglycerides, natural and synthetic fat bodies, preferably esters of fatty acids with alcohols of low carbon number, for example with isopropanol, propylene glycol or glycerol, or esters of fatty alcohols with alkanoic acids of low carbon number or with fatty acids, or from the group of the alkyl benzoates, and natural or synthetic hydrocarbon oils.

Useful substances include triglycerides of linear or branched, saturated or unsaturated, optionally hydroxylated, C₈-C₃₀ fatty acids, especially vegetable oils such as sunflower oil, corn oil, soya oil, rice oil, jojoba oil, babusscu oil, pumpkin oil, grape kernel oil, sesame oil, walnut oil, apricot oil, orange oil, wheat kernel oil, peach kernel oil, macadamia oil, avocado oil, sweet almond oil, lady's smock oil, castor oil, olive oil, peanut oil, rapeseed oil and coconut oil, and also synthetic triglyceride oils, for example the commercial product Myritol® 318. Hydrogenated triglycerides are also preferred in accordance with the invention. Oils of animal origin, for example bovine tallow, perhydrosqualene, lanolin, can also be used.

A further class of oil bodies preferred in accordance with the invention is that of the benzoic esters of linear or branched C₈-C₂₂ alcohols, for example the commercial products Finsolv® SB (isostearyl benzoate), Finsolv® TN (C₁₂-C₁₅-alkyl benzoate) and Finsolv® EB (ethylhexyl benzoate).

A further class of oil bodies preferred in accordance with the invention is that of the dialkyl ethers having a total of 12 to 36 carbon atoms, especially having 12 to 24 carbon atoms, for example di-n-octyl ether (Cetiol® OE), di-n-nonyl ether, di-n-decyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether, di-3-ethyldecyl ether, tert-butyl n-octyl ether, isopentyl n-octyl ether and 2-methylpentyl n-octyl ether, and also di-tert-butyl ether and diisopentyl ether.

Likewise useful are branched saturated or unsaturated fatty alcohols having 6-30 carbon atoms, for example isostearyl alcohol, and Guerbet alcohols.

A further class of oil bodies preferred in accordance with the invention is that of alkyl hydroxycarboxylates. Preferred alkyl hydroxycarboxylates are full esters of glycolic acid, lactic acid, malic acid, tartaric acid or citric acid. Further esters of hydroxycarboxylic acids suitable in principle are esters of β-hydroxypropionic acid, of tartronic acid, of D-gluconic acid, saccharic acid, mucic acid or glucuronic acid. Suitable alcohol components of these esters are primary, linear or branched aliphatic alcohols having 8 to 22 carbon atoms. Particular preference is given to the esters of C₁₂-C₁₅ fatty alcohols. Esters of this type are commercially available, for example under the Cosmacol® trade name from EniChem, Augusta Industriale.

A further class of oil bodies preferred in accordance with the invention is that of dicarboxylic esters of linear or branched C₂-C₁₀-alkanols, such as di-n-butyl adipate (Cetiol® B), di(2-ethylhexyl) adipate and di(2-ethylhexyl) succinate, and also diol esters such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate, and also diisotridecyl azelate.

Likewise preferred oil bodies are symmetric, unsymmetric or cyclic esters of carbonic acid with fatty alcohols, glyceryl carbonate or dicaprylyl carbonate (Cetiol® CC).

A further class of oil bodies preferred in accordance with the invention is that of the esters of dimers of unsaturated C₁₂-C₂₂ fatty acids (dimer fatty acids) with monohydric linear, branched or cyclic C₂-C₁₈-alkanols or with polyhydric linear or branched C₂-C₆-alkanols.

A further class of oil bodies preferred in accordance with the invention is that of hydrocarbon oils, for example those having linear or branched, saturated or unsaturated C₇-C₄₀ carbon chains, for example Vaseline, dodecane, isododecane, cholesterol, lanolin, synthetic hydrocarbons such as polyolefins, especially polyisobutene, hydrogenated polyisobutene, polydecane, and also hexadecane, isohexadecane, paraffin oils, isoparaffin oils, for example the commercial products of the Permethyl® series, squalane, squalene, and alicyclic hydrocarbons, for example the commercial product 1,3-di(2-ethylhexyl)cyclohexane (Cetiol® S), ozokerite and ceresin.

Available silicone oils and silicone waxes are preferably dimethylpolysiloxanes and cyclomethicones, polydialkylsiloxanes R₃SiO(R₂SiO)ₓSiR₃ where R is methyl or ethyl, more preferably methyl, and x is a number from 2 to 500, for example the dimethicones available under the trade names VICASIL (General Electric Company), DOW CORNING 200, DOW CORNING 225, DOW CORNING 200 (Dow Corning Corporation), and also the dimethicones available as SilCare® Silicone 41M65, SilCare® Silicone 41M70, SilCare® Silicone 41M80 (Clariant); stearyldimethylpolysiloxane, C₂₀-C₂₄-alkyldimethylpolysiloxane, C₂₄-C₂₈-alkyldimethylpolysiloxane, but also the methicones available as SilCare® Silicone 41M40, SilCare® Silicone 41M50 (Clariant), and also trimethyl-siloxysilicates [(CH₂)₃SiO)_{1/2}]ₓ[SiO₂]_{y}, where x is a number from 1 to 500 and y is a number from 1 to 500, dimethiconols R₃SiO[R₂SiO]ₓSiR₂OH and HOR₂SiO[R₂SiO]ₓSiR₂OH, where R is methyl or ethyl and x is a number up to 500, polyalkylarylsiloxanes, for example the polymethylphenylsiloxanes available under the trade names SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones and amino-, fatty acid-, alcohol-, polyether-, epoxy-, fluorine- and/or alkyl-modified silicone compounds, and also polyether siloxane copolymers.

Suitable cationic polymers are those known by the INCI name “Polyquaternium”, especially Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11 and Polyquaternium 37&mineral oil&PPG trideceth (Salcare SC95), PVP-dimethylaminoethyl methacrylate copolymer, guar hydroxypropyl triammonium chlorides, and also calcium alginate and ammonium alginate. In addition, it is possible to use cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, for example amidomethicones; copolymers of adipic acid and dimethylaminohydroxypropyldiethylenetriamine; polyaminopolyamide and cationic chitin derivatives, for example chitosan.

The inventive compositions may contain one or more of the abovementioned cationic polymers in amounts of 0.1% to 5.0% by weight, preferably of 0.2% to 3.0% by weight and more preferably of 0.5% to 2.0% by weight, based on the finished compositions.

In addition, the inventive compositions may comprise film formers which, according to the end use, are selected from salts of phenylbenzimidazolesulfonic acid, water-soluble polyurethanes, for example C₁₀-polycarbamyl polyglyceryl ester, polyvinyl alcohol, polyvinylpyrrolidone copolymers such as PVP/hexadecene or PVP/eicosene copolymer, for example vinylpyrrolidone/vinyl acetate copolymer, water-soluble acrylic acid polymers/copolymers and esters or salts thereof, for example partial ester copolymers of acrylic acid/methacrylic acid and polyethylene glycol ethers of fatty alcohols, such as Acrylate/Steareth-20-Methacrylate Copolymer, water-soluble cellulose, for example hydroxymethylcellulose, hydroxyethylcellulose, hydroxypropylcellulose, water-soluble quaterniums, polyquaterniums, carboxyvinyl polymers, such as carbomers and salts thereof, polysaccharides, for example polydextrose and glucan, vinyl acetate/crotonate, for example available under the trade name Aristoflex® A 60 (Clariant), and polymeric amine oxides, for example representatives obtainable under the trade names Diaformer Z-711, 712, 731, 751.

The inventive compositions may contain one or more film formers in amounts of 0.1% to 10.0% by weight, preferably of 0.2% to 5.0% by weight and more preferably of 0.5% to 3.0% by weight, based on the finished compositions.

The desired viscosity of the compositions can be established by adding thickeners and gelating agents. Useful substances are preferably cellulose ethers and other cellulose derivatives (e.g. carboxymethyl cellulose, hydroxyethyl cellulose), gelatin, starch and starch derivatives, sodium alginates, fatty acid polyethylene glycol esters, agar agar, tragacanth or dextrin derivatives, especially dextrin esters. Additionally suitable are metal salts of fatty acids, preferably having 12 to 22 carbon atoms, for example sodium stearate, sodium palmitate, sodium laurate, sodium arachidate, sodium behenate, potassium stearate, potassium palmitate, sodium myristate, aluminum monostearate, hydroxyl fatty acids, for example 12-hydroxystearic acid, 16-hydroxyhexadecanoyl acid; fatty acid amides; fatty acid alkanolamides; dibenzalsorbitol and alcohol-soluble polyamides and polyacrylamides or mixtures of such. It is also possible to use crosslinked and uncrosslinked polyacrylates such as carbomers, sodium polyacrylates or polymers containing sulfonic acid, such as ammonium acryloyldimethyltaurateNP copolymer.

The inventive compositions preferably contain from 0.01% to 10.0% by weight, more preferably from 0.1% to 5.0% by weight, especially preferably from 0.2% to 3.0% by weight and most preferably from 0.4% to 2.0% by weight of thickeners and/or gelating agents.

Superfatting agents used may preferably be lanolin and lecithin, nonethoxylated and polyethoxylated or acylated lanolin derivatives and lecithin derivatives, polyol fatty acid esters, mono-, di- and triglycerides and/or fatty acid alkanolamides, where the latter simultaneously serve as foam stabilizers, which are preferably used in amounts of 0.01% to 10.0% by weight, more preferably of 0.1% to 5.0% by weight and especially preferably of 0.5% to 3.0% by weight.

Active antimicrobial ingredients used are cetyltrimethylammonium chloride, cetylpyridinium chloride, benzethonium chloride, diisobutylethoxyethyldimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, for example L-lysine hexadecylamide, citrate heavy metal salts, salicylates, piroctoses, especially zinc salts, pyrithiones and heavy metal salts thereof, especially zinc pyrithione, zinc phenolsulfate, farnesol, ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, selenium disulfide and Octopirox, iodopropynyl butylcarbamate, methylchloroisothiazolinone, methylisothiazolinone, methyldibromoglutaro-nitrile, AgCl, chloroxylenol, sodium salt of diethylhexyl sulfosuccinate, sodium benzoate, and phenoxyethanol, benzyl alcohol, phenoxyisopropanol, parabens, preferably butyl, ethyl, methyl and propyl paraben, and sodium salts thereof, pentanediol, 1,2-octanediol, 2-bromo-2-nitropropane-1,3-diol, ethylhexylglycerol, benzyl alcohol, sorbic acid, benzoic acid, lactic acid, imidazolidinylurea, diazolidinylurea, dimethyloldimethylhydantoin (DMDMH), sodium salt of hydroxymethylglycinate, hydroxyethylglycine of sorbic acid and combinations of these active substances.

The inventive compositions contain the active antimicrobial ingredients preferably in amounts of 0.001% to 5.0% by weight, more preferably of 0.01% to 3.0% by weight and especially preferably of 0.1% to 2.0% by weight, based on the finished compositions.

The inventive compositions may further comprise active biogenic ingredients selected from plant extracts, for example aloe vera, and also local anesthetics, antibiotics, antiphlogistics, antiallergics, corticosteroids, sebostatics, Bisabolol®, Allantoin®, Phytantriol®, proteins, vitamins selected from niacin, biotin, vitamin B2, vitamin B3, vitamin B6, vitamin B3 derivatives (salts, acids, esters, amides, alcohols), vitamin C and vitamin C derivatives (salts, acids, esters, amides, alcohols), preferably as sodium salt of the monophosphoric acid ester of ascorbic acid or as magnesium salt of the phosphoric acid ester of ascorbic acid, tocopherol and tocopherol acetate, and also vitamin E and/or derivatives thereof.

The inventive compositions may contain active biogenic ingredients preferably in amounts of 0.001% to 5.0% by weight, more preferably 0.01% to 3.0% by weight and especially preferably 0.1% to 2.0% by weight, based on the finished compositions.

The inventive compositions may contain astringents, preferably magnesium oxide, aluminum oxide, titanium dioxide, zirconium dioxide and zinc oxide, oxide hydrates, preferably aluminum oxide hydrate (boehmite) and hydroxides, preferably of calcium, magnesium, aluminum, titanium, zirconium or zinc, and also aluminum chlorohydrates, preferably in amounts of from 0% to 50.0% by weight, more preferably in amounts of from 0.01% to 10.0% by weight and especially preferably in amounts of from 0.1% to 10.0% by weight. Preferred deodorizing substances are allantoin and bisabolol. These are preferably used in amounts of from 0.0001% to 10.0% by weight.

Examples of moisture donors available are isopropyl palmitate, glycerol and/or sorbitol.

Stabilizers used may be metal salts of fatty acids, for example magnesium stearate, aluminum stearate and/or zinc stearate, preferably in amounts of 0.1% to 10.0% by weight, preferably of 0.5% to 8.0% by weight and more preferably of 1.0% to 5.0% by weight, based on the finished compositions.

Suitable preservatives are the preservatives listed in the relevant annex of the European cosmetics legislation, for example phenoxyethanol, benzyl alcohol, parabens, benzoic acid and sorbic acid; a particularly suitable example is 1,3-bis(hydroxymethyl)-5,5-dimethylimidazolidine-2,4-dione (Nipaguard® DMDMH).

Preferentially suitable pearlizing components are fatty acid monoalkanolamides, fatty acid dialkanolamides, monoesters or diesters of alkylene glycols, especially ethylene glycol and/or propylene glycol or oligomers thereof, with higher fatty acids, for example palmitic acid, stearic acid and behenic acid, monoesters or polyesters of glycerol with carboxylic acids, fatty acids and metal salts thereof, ketosulfones or mixtures of the specified compounds. Particular preference is given to ethylene glycol distearates and/or polyethylene glycol distearates having an average of 3 glycol units.

If the inventive compositions contain pearlizing compounds, they are preferably present in the inventive compositions in an amount of 0.1% to 15.0% by weight and more preferably in an amount of from 1.0% to 10.0% by weight.

Fragrance and/or perfume oils used may be individual odorant compounds, for example the synthetic products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl propionate, styrallyl propionate and benzyl salicylate. The ethers include, for example, benzyl ethyl ethers, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, alpha-isomethylionone and methyl cedryl ketone, the alcohols include anethole, citronellol, eugenol, geraniol, linalool, phenylethyl alcohol and terpineol, and the hydrocarbons include primarily the terpenes and balsams. Preference is given to using mixtures of different odorants which together produce a pleasing scent note.

Perfume oils may also include natural odorant mixtures, as obtainable from vegetable or animal sources, for example pine oil, citrus oil, jasmine oil, lily oil, rose oil or ylang-ylang oil. Essential oils of relatively low volatility, which are usually used as aromatic components, are also suitable as perfume oils, for example sage oil, chamomile oil, clove oil, melissa oil, mint oil, cinnamon leaf oil, linden blossom oil, juniperberry oil, vetiver oil, olibanum oil, galbanum oil and ladanum oil.

The inventive compositions are preferably adjusted to a pH in the range from 2 to 12, preferably in the range from 3 to 9.

Acids or alkalis used for adjusting the pH are preferably mineral acids, especially HCl, inorganic bases, especially NaOH or KOH, or organic acids, especially citric acid.

The total proportion of further additives of component (d) in the products or in the inventive compositions is preferably from 1.0% to 25.0% by weight and more preferably from 2.0% to 20.0% by weight.

In a preferred embodiment of the invention, the inventive compositions are cosmetic, dermatological or pharmaceutical compositions.

Preferably, the inventive cosmetic, dermatological or pharmaceutical compositions are care products for the skin and hair treatment products.

Examples of preferred inventive cosmetic, dermatological or pharmaceutical compositions are skincare products such as creams, lotions, day creams, night creams, care creams, nutrient creams, body lotions and ointments.

In a further preferred embodiment of the invention, the inventive compositions are oil-in-water emulsions, preferably oil-in-water emulsions for treatment or care of the skin.

In a particularly preferred embodiment of the invention, the inventive compositions are compositions for treatment or care of hair, especially hair treatment products such as rinse-off hair conditioners, cream rinses, clear rinses, hair cures, hair colorants and hair tints, permanent wave products, hair conditioners in aerosol, spray and fluid form.

The inventive compositions, especially the inventive cosmetic, dermatological or pharmaceutical compositions, are advantageously suitable for treatment or care of the skin.

The present invention therefore further provides for the use of the inventive compositions, especially of the inventive cosmetic, dermatological or pharmaceutical compositions, for treatment or care of the skin.

The inventive compositions, especially the inventive cosmetic, dermatological or pharmaceutical compositions, are also advantageously suitable for treatment or care of hair.

The present invention therefore further provides for the use of the inventive compositions, especially of the inventive cosmetic, dermatological or pharmaceutical compositions, for treatment or care of hair.

Preference is given to the use of the inventive cosmetic, dermatological or pharmaceutical compositions as hair treatment products, especially for conditioning of hair.

The examples which follow serve to illustrate the invention, but without restricting it thereto.

### EXAMPLES

The percentages stated in the examples are percent by weight (% by weight), unless explicitly stated otherwise.

The N-acyl-N-methylglucamines described hereinafter were prepared according to EP 0 550 637 from the corresponding fatty acid methyl esters and N-methylglucamide in the presence of 1,2-propylene glycol as solvent, and were obtained in solid form consisting of active substance, i.e. N-methyl-N-acylglucamines, and 1,2-propylene glycol.

| | | Active | | |
|---|---|---|---|---|
| Preparation | | substance | 1,2-Propylene | |
| example | Methyl ester | (%) | glycol (%) | Melting point |
| 1 | C12/14 | 90 | 10 | 85 |
| 2 | C16/18 | 80 | 20 | 65 |
| 3 | C20/22 | 80 | 20 | 120 |

The viscosities were measured with a Brookfield DV II viscometer, the spindles from the RV spindle set at 20 revolutions/minute and 20° C. Spindles 1 to 7 from the RV spindle set are used. Under these measurement conditions, spindle 1 is chosen for viscosities of not more than 500 mPa·s, spindle 2 for viscosities of not more than 1000 mPa·s, spindle 3 for viscosities of not more than 5000 mPa·s, spindle 4 for viscosities of not more than 10 000 mPa·s, spindle 5 for viscosities of not more than 20 000 mPa·s, spindle 6 for viscosities of not more than 50 000 mPa·s and spindle 7 for viscosities of not more than 200 000 mPa·s.

The cream rinse products which follow were produced from emulsifier, cetearyl alcohol and behentrimonium chloride (Genmin® KDMP, Clariant).

### Production:

Emulsifier (amount used according to active substance, 1.5%) and cetearyl alcohol (3%) were melted at 80° C.; in parallel, the behentrimonium chloride (1% active substance) was dissolved in water at 80° C. The two phases were combined while stirring and the resultant cream rinse was cooled while stirring and the pH was adjusted to pH=4.0 with citric acid.

| | | Viscosity | |
|---|---|---|---|
| Example | Emulsifier | at 20° C. | Appearance |
| Comparative | Ceteareth-3 | 5500 | white emulsion |
| example 1 | | | |
| Comparative | Preparation example 1 | 900 | white, low-viscosity |
| example 2 | | | emulsion |
| Example 1 | Preparation example 2 | 7400 | white emulsion |
| Comparative | Preparation example 3 | 7100 | white emulsion with |
| example 3 | | | inhomogeneities |

The products were assessed by a trained sensory panel having 5 participants with respect to the parameters specified below. ++=very good, +=good, o=satisfactory, −=adequate, −−=unsatisfactory assessed, and the parameters, after forming the average, assessed qualitatively against the standard (comparative example 1) assessed. This involved, after washing the hair in a half-and-half test, applying two of the test products to one half of the head each, rinsing out with water and assessing the hair properties during the rinsing, during the drying and when dry.

| | Smoothness of hair on | Feel of hair | |
|---|---|---|---|
| Example | rinsing | during drying | Feel of hair when dry |
| Comparative | o (standard) | o (standard) | o (standard) |
| example 1 | | | |
| Comparative | o (no improvement | + (somewhat | o (no improvement from |
| example 2 | from standard) | softer) | standard) |
| Example 1 | + (soft, good | ++ (softer) | + (better than comparative |
| | detangling) | | example 1 at the hair tips) |
| Comparative | inhomogeneous | inhomogeneous | inhomogeneous emulsion |
| example 3 | emulsion | emulsion | |

The inventive compositions (example 1) show distinctly improved viscosities compared to the prior art (Henkel C12/14 glucamide, comparative example 2). Moreover, the conditioning properties are distinctly improved compared to the comparative examples when the inventive C16-18 N-acyl-N-methylglucamines are used; specifically, the softness of the hair during the drying phase is improved.

1. A composition comprising
a) one or more N-methyl-N-acylglucamines, where at least 50% by weight of the N-methyl-N-acylglucamines have a C₁₆-, C₁₇- and/or C₁₈-acyl group,
b) one or more fatty alcohols,
c) one or more monomeric cationic surfactants,
d) optionally further additives and
e) water.

2. The composition as claimed in claim 1, wherein at least 50% by weight of the N-methyl-N-acylglucamines are compounds of the formula (I) 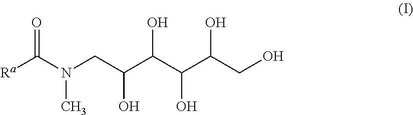
where R^{a}—CO is derived from palmitic acid, stearic acid, oleic acid or linoleic acid.

3. The composition as claimed in claim 1, comprising
(a) 0.1% to 5% by weight of component (a),
(b) 0.1% to 10% by weight of component (b),
(c) 0.1% to 5% by weight of component (c),
(d) 0% to 30% by weight of component (d) and
(e) 99.8% to 50% by weight of component (e),
where the sum total of components (a), (b), (c), (d) and (e) is 100% by weight.

4. The composition as claimed in claim 1, comprising
(a) 0.2% to 3% by weight of component (a),
(b) 0.1% to 10% by weight of component (b),
(c) 0.1% to 3% by weight of component (c),
(d) 0% to 30% by weight of component (d) and
(e) 99.6% to 54% by weight of component (e),
where the sum total of components (a), (b), (c), (d) and (e) is 100% by weight.

5. The composition as claimed in claim 1, wherein component a) contains at least 80% by weight of N-methyl-N-acylglucamines having a C₁₆-C₁₇- and/or C₁₈-acyl group.

6. The composition as claimed in claim 1, any of claims 1 to 5, comprising one or more cationic surfactants of the formula (II) 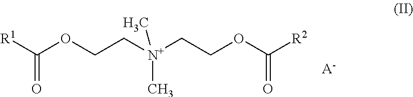
in which
R¹CO and R²CO are each independently linear or branched saturated acyl groups having 18 to 24 carbon atoms,
A⁻ is a counterion
and the total amount of C₁₈₋₂₃-alkyl-COO— groups, based on all the R¹COO— and R²COO— groups, is 40.0% by weight or more.

7. The composition as claimed in claim 1, comprising one or more cationic surfactants of the formula (III)
CₙH₂ₙN⁺(CH₃)₃X⁻ (III)
where n is 16, 18, 20 or 22 and X⁻ is a chloride ion or methosulfate ion.

8. The composition as claimed in claim 1, comprising one or more cationic surfactants of the formula (IV)
[R¹CO—NH—CH₂—CH₂—CH₂—NR²R³R⁴]⁺X⁻ (IV)
where R¹CO is a linear or branched, saturated or unsaturated acyl radical having 16 to 32 carbon atoms, R² and R³ are each methyl, R⁴ is H or methyl and X⁻ is an anion selected from the group consisting of chloride, methosulfate, lactate, citrate, glutamate and glycolate.

9. The composition as claimed in claim 1, wherein component c) comprises one or more fatty alcohols selected from the group consisting of lauryl alcohol, stearyl alcohol, cetyl alcohol, Guerbet alcohol and behenyl alcohol.

10. A cosmetic, dermatological or pharmaceutical composition comprising the composition as claimed in claim 1.

11. The composition as claimed in claim 10 in the form of an emulsion.

12. The cosmetic composition as claimed in claim 10 in the form of a hair treatment composition.

13. The composition as claimed in claim 10, in the form of a cream or lotion.

14. At least one N methyl-N-acylglucamine as claimed in claim 1 for thickening emulsions, wherein the at least one N-methyl-N-acylglucamine a), is combined with at least one fatty alcohol b) and the ratio of components a):b) is 1:9 to 5:5.
